SEQUENCE LISTING (10 sequences)

SEQ ID NO: 1 - DNA, 20 bp, Artificial
  The forward primer used for detecting mRNA for IL-6.
cctgcaagaccatcgacatg

SEQ ID NO: 2 - DNA, 20 bp, Artificial
  The reverse primer used for detecting mRNA for IL-6.
ctggcgagccttagtttgga

SEQ ID NO: 3 - DNA, 24 bp, Artificial
  The forward primer used for detecting mRNA for CFB.
tggtttgggaacacaggaagggta

SEQ ID NO: 4 - DNA, 20 bp, Artificial
  The reverse primer used for detecting mRNA for CFB.
ctggcgagccttagtttgga

SEQ ID NO: 5 - DNA, 20 bp, Artificial
  The forward primer used for detecting mRNA for STAT3.
gatccagtccgtggaaccat

SEQ ID NO: 6 - DNA, 23 bp, Artificial
  The reverse primer used for detecting mRNA for STAT3.
atagcccatgatgatttcagcaa

SEQ ID NO: 7 - DNA, 18 bp, Artificial
  The forward primer used for detecting mRNA for JAK2.
gctcagtggcggcatgat

SEQ ID NO: 8 - DNA, 21 bp, Artificial
  The reverse primer used for detecting mRNA for JAK2.
cactgccatcccaagacattc

SEQ ID NO: 9 - DNA, 19 bp, Artificial
  The forward primer used for detecting mRNA for GAPDH.
aacagcgacacccatcctc

SEQ ID NO: 10 - DNA, 24 bp, Artificial
  The reverse primer used for detecting mRNA for GAPDH.
cataccaggaaatgagcttgacaa

(19) United States
(12) Patent Application Publication
(10) Pub. No.: US 2012/0245108 A1
(43) Pub. Date: Sep. 27, 2012

(54) METHOD OF MODULATING COMPLEMENT FACTOR B (CFB) EXPRESSION IN CELLS

(75) Inventors: Suh-Hang Hank Juo, Kaohsiung City (TW); Wen-Wen Chou, Kaohsiung City (TW); Jing-Mei Wu, Yunlin County (TW); Chung-Ling Liang, Kaohsiung City (TW)

(73) Assignee: KAOHSIUNG MEDICAL UNIVERSITY, Kaohsiung City (TW)

(21) Appl. No.: 13/069,619

(22) Filed: Mar. 23, 2011

(51) Int. Cl. A61K 31/7034; A61P 9/10; A61P 25/18; C12N 5/071; A61P 31/00; A61P 31/10; A61P 1/02; A61P 27/02; A61P 37/00

(52) U.S. Cl. 514/25; 435/375

(57) ABSTRACT

The present invention relates to a method of modulating complement factor B (CFB) expression in cells, comprising administering an effective amount of tannic acid to the cells. This method can be applied in treating or prophylaxis of the disease, disorder or medical condition associated to complement factor B (CFB) expression.

### FIELD OF THE INVENTION

The present invention relates to a method of modulating complement factor B (CFB) expression in cells.

### BACKGROUND OF THE INVENTION

In recent years, the destruction of the ozone layer raises the levels of solar ultraviolet (UV) radiation that threatens human health. UV radiation forms part of the electromagnetic spectrum with wavelengths between 200 nm and 400 nm. UV radiation can be divided into three categories dependent on the wavelengths: long wave UVA (320-400 nm), medial wave UVB (280-320 nm), and short wave UVC (200-280 nm). UV radiation can damage the ocular tissues (Galichanin K, Löfgren S, Bergmanson J, Söderberg P. Evolution of damage in the lens after in vivo close to threshold exposure to UV-B radiation: cytomorphological study of apoptosis. Exp Eye Res. 2010 September; 91(3):369-77) and has a phototoxic effect on the retinal pigment epithelium (RPE) cells (Patton W P, Chakravarthy U, Davies R J, Archer D B. Comet assay of UV-induced DNA damage in retinal pigment epithelial cells. Invest Ophthalmol Vis Sci 1999; 40:3268-3275). The RPE is the site of primary injury by near-UV (300-400 nm) radiation and UV-induced damage may be associated with age-related macular degeneration (AMD) (Szaflik J P, Janik-Papis K, Synowiec E, et al. DNA damage and repair in age-related macular degeneration. Mutat Res 2009; 669:169-176).

AMD is the most frequent cause of irreversible blindness in the elder (Almeida L N, Carolino R M, Sperandio D C, Nehemy M B, De Marco L A. The role of molecular genetic factors in age-related macular degeneration. Arq Bras Oftalmol 2009; 72:567-572). Previous studies have shown that complement factors B (CFB) and H (CFH) play an important role in the pathogenesis of AMD in more than 50% of cases (Maller J, George S, Purcell S, et al. Common variation in three genes, including a noncoding variant in CFH, strongly influences risk of age-related macular degeneration. Nat Genet 2006; 38:1055-1059; Gold B, Merriam J E, Zernant J, et al. Variation in factor B (BF) and complement component 2 (C2) genes is associated with age-related macular degeneration. Nat Genet 2006; 38:458-462). CFB and CFH are involved in body's immune and inflammatory response. Pro-inflammatory cytokines are important mediators of cellular activities for several ocular diseases. For example, pro-inflammatory cytokine interleukin-6 (IL-6) plays a role in inflammation of RPE (Qin S, Ni M, De Vries G W. Implication of S-adenosylhomocysteine hydrolase in inhibition of TNF-alpha and IL-1beta-induced expression of inflammatory mediators by AICAR in RPE cells. Invest Ophthalmol Vis Sci 2008; 49:1274-1281). UVB radiation can induce the IL-6 expression that causes intracellular signaling in cultured human pterygium epithelial cells (Di Girolamo N, Wakefield D, Coroneo M T. UVB-mediated induction of cytokines and growth factors in pterygium epithelial cells involves cell surface receptors and intracellular signaling. Invest Ophthalmol Vis Sci 2006; 47:2430-2437). IL-6 can bind to its receptor (such as gp130) to activate the Janus kinase (JAK) and signal transducer and activator of transcription 3 (STAT-3) pathway to induce expression of several downstream target genes (Murray P J. The JAK-STAT signaling pathway: input and output integration. J Immunol 2007; 178:2623-2629). It has been reported that IL-6 increases the rate of synthesis of CHB mRNA and proteins in fibroblasts (Katz Y, Revel M, Strunk R C. Interleukin 6 stimulates synthesis of complement proteins factor B and C3 in human skin fibroblasts. Eur J Immunol 1989; 19:983-988).

United States Publication No. 2010/0247434 discloses a method of ameliorating or preventing angiogenesis in a mammal comprising administering to the mammal a therapeutically effective amount of gallic acid or its derivatives. Although indicating that angiogenic-associated disease includes age-related macular degeneration and gallic acid derivative includes tannic acid, its experiments do not show the effect of tannic acid on treating macular degeneration. In addition, US 2010/0247434 does not disclose the effect of tannic acid on modulating complement factor B expression.

It remains unclear which signal pathway is involved in UVB-induced IL-6 in RPE.

### BRIEF DESCRIPTION OF THE DRAWINGS

FIG. 1 shows the effects of UVB radiation on cell viability. ARPE-19 cells (2×10⁵ cells) in 3-cm culture dish were incubated for 24 hours. Cells were treated with either 0 mJ/cm² (open bars) or 5-25 mJ/cm² UVB (gray bars) then incubated for 24 hours. Cell number was measured with trypan blue exclusion assay. The results are shown as the mean of three independent experiments. *P<0.05 vs. 0 mJ/cm² UVB.

FIG. 2 shows that UVB radiation up-regulates IL-6/JAK2/STAT3 mRNA expression. ARPE-19 cells were treated with either 0 mJ/cm² (open bars) or 5-15 mJ/cm² UVB (gray bars) then incubated for 24 hours. The relative expression levels of IL-6/JAK2/STAT3 mRNA were assessed by real-time RT-PCR. UVB induced IL-6(A)/JAK2(B)/STAT3(C) mRNA expression. The results normalizated against to GAPDH are shown as the mean of three independent experiments. *P<0.05, ** P<0.01, *** P<0.001 vs. 0 mJ/cm² UVB.

FIG. 3 shows that tannic acid attenuates UVB-induced IL-6 up-regulation. ARPE-19 cells were treated with either 0 mJ/cm² (open bars) or 5-15 mJ/cm² UVB (gray bars) then incubated for 24 hours. The protein expression levels of IL-6 were assessed by ELISA. UVB dose-dependently induced IL-6 protein production (A). TA (25 μM, bar 4) attenuated UVB (bar 2)-induced IL-6 protein production (B). The results are shown as the mean of three independent experiments. *P<0.05 and **P<0.01 vs. 0 mJ/cm² UVB; ^{#}P<0.05 vs. 10 mJ/cm² UVB.

FIG. 4 shows that tannic acid suppresses UVB-induced phosphorylation of STAT3 on Tyr705. ARPE-19 cells (5×10⁵ cells) in 6-cm culture dish were incubated for 24 hours. Cells were exposed to UVB radiation at indicated doses then incubated for 24 hours. Phospho-STAT3^{Tyr705} was detected by immunoblotting. UVB increased phospho-STAT3^{Tyr705} levels (lane 2) and TA (25 μM) attenuated UVB-induced phospho-STAT3^{Tyr705} (lane 4).

FIG. 5 shows that tannic acid and JAK2 inhibitor attenuates UVB-induced CFB mRNA. ARPE-19 cells were treated with either 0 mJ/cm² (open bars) or 5-15 mJ/cm² UVB (gray bars) then incubated for 24 h. CFB mRNA expression was assessed by real-time RT-PCR. UVB induced CFB mRNA (A). TA (25 μM) and AG490 (40 μM in 0.2% DMSO) attenuated UVB-induced CFB mRNA (B-C). The relative expression data were presented after normalization against to GAPDH. The results are shown as the mean of three independent experiments. *P<0.05 **P<0.01 and ***P<0.001 vs. 0 mJ/cm² UVB; ^{#}P<0.05 and ^{##}P<0.01 vs. 10 mJ/cm² UVB.

FIG. 6 shows schematic representation of the signaling pathways involved in the UVB-induced RPE inflammation.

### SUMMARY OF THE INVENTION

The present invention relates to a method of modulating complement factor B (CFB) expression in cells, comprising administering an effective amount of tannic acid to the cells.

### DETAILED DESCRIPTION OF THE INVENTION

The present invention is to investigate whether the IL-6/JAK2/STAT3 signaling pathway can be activated by UVB (Ultraviolet B), and whether activation of this pathway can in turn cause CFB (complement factor B) up-regulation in RPE (retinal pigment epithelium) cells. Tannic acid (TA) is also tested as a protective agent from UVB damage to the RPE cells. TA's anti-inflammatory mechanism is also explored in the present invention.

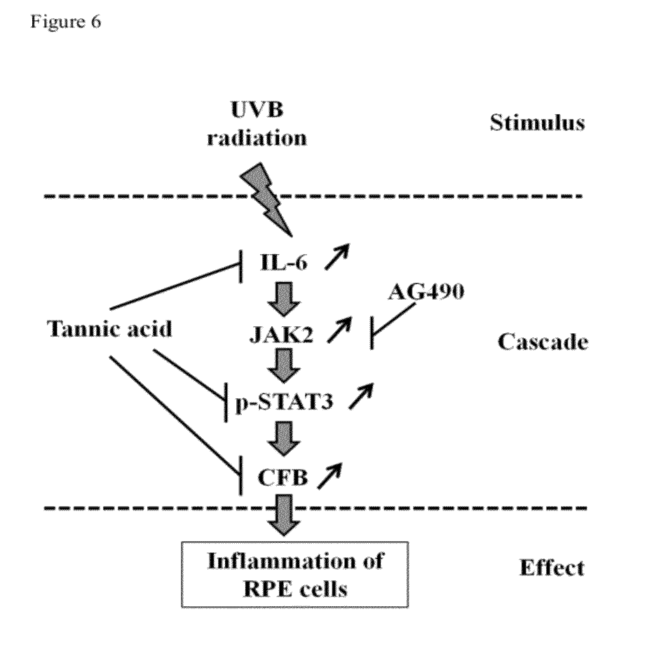

The present invention finds that UVB triggers the IL-6 protein expression, which in turn activates the signal transduction in the IL-6/JAK2/STAT3/CFB pathway leading to up-regulation of CFB (FIG. 6). This signaling pathway explains the UV-induced AMD (age-related macular degeneration) pathogenesis. Because an increased expression of CFB in the RPE cells has been linked to the development of AMD (Maller J, George S, Purcell S, et al. Common variation in three genes, including a noncoding variant in CFH, strongly influences risk of age-related macular degeneration. Nat Genet 2006; 38:1055-1059; Gold B, Merriam J E, Zernant J, et al. Variation in factor B (BF) and complement component 2 (C2) genes is associated with age-related macular degeneration. Nat Genet 2006; 38:458-462), the results of the present invention provide more insight to UV-induced AMD. The present invention further demonstrated that TA can efficiently prevent the UVB damage by inhibiting IL-6 production, STAT3 phosphorylation and CFB up-regulation. Therefore, the present invention illustrates one of the detrimental mechanisms of UVB on the RPE cells, and also show a potential effect of TA to prevent UVB damages. The present invention also found that TA can reduce STAT3 phosphorylation and CFB expression even without UV radiation, which increases TA application to prevent AMD development.

Component factor B is involved in acute phase inflammatory reaction and its serum levels change significantly in the course of inflammation. CFB is a centrally important component of the alternative complement pathway. Alternative pathway activation results in factor B cleavage leading to the production of Ba and Bb fragments. Both Ba and Bb fragments have been reported to express a variety of biological activities. Several diseases have been linked to CFB such as age-related macular degeneration, complement factor B deficiency, hemolytic uremic syndrome, atherosclerosis, schizophrenia, glomerulonephritis, autoimmune diseases including but not limited to Behçet's syndrome, recurrent oral ulceration and Crohn's disease, uveitis, infectious diseases or fungal infection.

The terms used in the description herein will have their ordinary and common meaning as understood by those skilled in the art, unless specifically defined otherwise. As used throughout the instant application, the following terms shall have the following meanings:

The term “UVB” or “ultraviolet B” refers to one of the three types of invisible light rays (together with ultraviolet A and ultraviolet C) given off by the sun.

The term “disease, disorder or medical condition associated to complement factor B (CFB) expression” refers to the disease, disorder or medical condition that is caused partially or mainly by inappropriate expression of complement factor B (CFB). The disease, disorder or medical condition associated to complement factor B (CFB) expression may includes but not limited to age-related macular degeneration, complement factor B deficiency, hemolytic uremic syndrome, atherosclerosis, schizophrenia, glomerulonephritis, autoimmune diseases such as Behçet's syndrome, recurrent oral ulceration and Crohn's disease, uveitis, infectious diseases or fungal infection.

Thus, the present invention provides a method of modulating complement factor B (CFB) expression in cells, comprising administering an effective amount of tannic acid to the cells. In a preferred embodiment, the method is applied in treating or prophylaxis of the disease, disorder or medical condition associated to complement factor B (CFB) expression, preferably, the disease, disorder or medical condition is selected from age-related macular degeneration, complement factor B deficiency, hemolytic uremic syndrome, atherosclerosis, schizophrenia, glomerulonephritis, autoimmune diseases, uveitis, infectious diseases or fungal infection. Preferably, the disease, disorder or medical condition associated to complement factor B (CFB) expression is age-related macular degeneration. In another preferred embodiment, the complement factor B (CFB) expression is induced by ultraviolet light, preferably by ultraviolet B. In a preferred embodiment, the cells are retinal pigment epithelium cells. In another preferred embodiment, the method modulates CFB expression by modulating STAT3 phosphorylation. Preferably, the method modulates STAT3 phosphorylation by modulating IL-6 protein production.

### EXAMPLES

The examples below are non-limiting and are merely representative of various aspects and features of the present invention.

### Example 1

### Cell Culture and Reagents

ARPE-19 (CRL-2302, human retinal pigment epithelial cells, American Type Culture Collection, Manassas, Va.) cells were cultured in 1:1 mixture of Dulbecco's modified Eagle's medium and HAM F-12 nutrient mixture supplemented with 10% fetal bovine serum at 37° C., 5% CO2, and 90% relative humidity. Confluent cells (90% confluence) were used throughout the experiments. Culture materials were obtained from Gibco-BRL (Rockville, Md.). Tannic acid, AG490 and all other reagents were obtained from Sigma Chemical Co. (St. Louis, Mo.).

### UVB Radiation

The source of UVB radiation was performed using the EL series of UV lamp (UVP Inc., San Gabriel, Calif., USA). One hundred percent energy emission was at wavelength 302 nm with a radiation intensity of 1.04 mW/cm² in a lamp and a target distance of 15 cm was measured by a UVX Digital Radiometer (UVP Inc.).

### Trypan Blue Exclusion Assay

Trypan blue exclusion assay was used to determine the effect of treatments on the growth and viability of ARPE-19 cells. ARPE-19 cells were plated about 200,000 cells in a 3-cm dish and cultured for 18-24 hours. Cells were exposed to different doses of UVB radiation for 24 hours. After 24 hours, cells were trypsinized and collected in the same tube. The cell pellet was washed and re-suspended in 300 μl PBS (pH 7.4). Trypan blue was added to an equivalent volume (μl) of cell suspension, and the number of cells was counted using a hemacytometer in duplicate for each sample.

### Real-Time Reverse Transcription Polymerase Chain Reaction (RT-PCR)

The total RNA was isolated from ARPE-19 cells and cells exposed to UVB radiation (5-15 mJ/cm²) for 24 hr using TRIzol Reagent (invitrogen). One μg of total RNA was subject to reverse transcriptase RT-PCR by using RT-PCR kit (Applied Biosystems). The primers used for detecting mRNA for IL-6 were 5′-CCTGCAAG ACCATCGACATG-3′ (forward, SEQ ID NO. 1) and 5′-CTGGCGAGCCTTAGTTTGGA-3′ (reverse, SEQ ID NO. 2); CFB were 5′-TGGTTTGGGAACACAGGAAGGGTA-3′ (forward, SEQ ID NO. 3) and 5′-TCCCTTTGAAGGGCGAATGACTGA-3 (reverse, SEQ ID NO. 4); STAT3 were 5′-GATCCAGTCCGTGGAACCAT-3′ (forward, SEQ ID NO. 5) and 5′-ATAGCCCATGATGATTTCAGCAA-3′ (reverse, SEQ ID NO. 6); JAK2 were 5′-GCTCAGTGGCGGCATGAT-3′ (forward, SEQ ID NO. 7) and 5′-CACTGCCATCCCAAGACATTC-3′ (reverse, SEQ ID NO. 8); GAPDH were 5′-AACAGCGACACCCATCCTC-3′ (forward, SEQ ID NO. 9) and 5′-CATACCAGGAAATGAGCTTGACAA-3′ (reverse, SEQ ID NO. 10). Real-time PCR was performed in a Gene Amp 7900® Sequence Detection System SDS (Applied Biosystems, Foster City, Calif., USA) using SYBR® Green PCR Master Mix Applied Biosystems. The relative gene expression is the comparative CT method. Each sample was normalized on the basis of its GAPDH content according to the formula 2^{-ΔΔCT}.

### Immunoblotting

Total cell lysates were resolved by 10% sodium dodecyl sulfate-polyacryl amide gel electrophoresis, transferred to membranes and blocked, and then probed phospho-STAT3(Tyr705) and STAT3 (Cell Signaling Technology, Danvers, Mass.) antibodies. To control for protein loading, all blots were probed with GAPDH (Millipore, Billerica, Mass.) as well. The bands were detected using the enhanced chemiluminescence system (Millipore, Billerica, Mass.).

### Enzyme-Linked Immunosorbent Assay (ELISA)

ARPE-19 cells were plated about 200,000 cells in a 3-cm dish and cultured for 18-24 hours. Cells were exposed to UVB radiation for 24 hours, 1 ml aliquots of cell culture supernatants were drawn off and frozen at −80° C. ELISAs were carried out with the OptEIA human IL-6 ELISA kit (BD Pharmingen) according to the manufacturer's instructions. Supernatant samples were plated in duplicate wells of a 96-well plate. Freshly diluted rIL-6 which is useful as a standard control was used to generate a standard curve on each plate analyzed. Plates were read at 450 nm using a spectrophotometric plate reader. Raw data were corrected against blank wells and converted to pg/ml using the standard curve. Each sample was normalized on its cell number.

### Statistical Analysis

Data of a continuous variable was expressed as the mean±standard deviation (SD). Unpaired t-tests were used to compare means between two groups. A p-value of less than 0.05 was considered statistically significant.

### Results

### Dose Effects of UVB Radiation on ARPE-19 Cell Growth

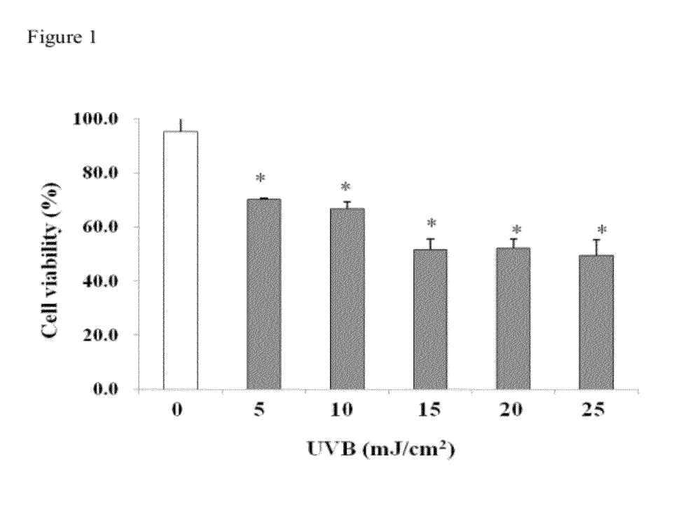

The UVB radiation dose-dependently (0-25 mJ/cm²) decreased cell proliferation at 24 h after radiation (FIG. 1). Since cells had a viability of 74-54% by the dose of UVB radiation (5-15 mJ/cm²), the UVB doses up to 15 mJ/cm² were decided to be used for further studies.

Effects of UVB Radiation on mRNA Expression of the IL-6/JAK2/STAT3 Pathway

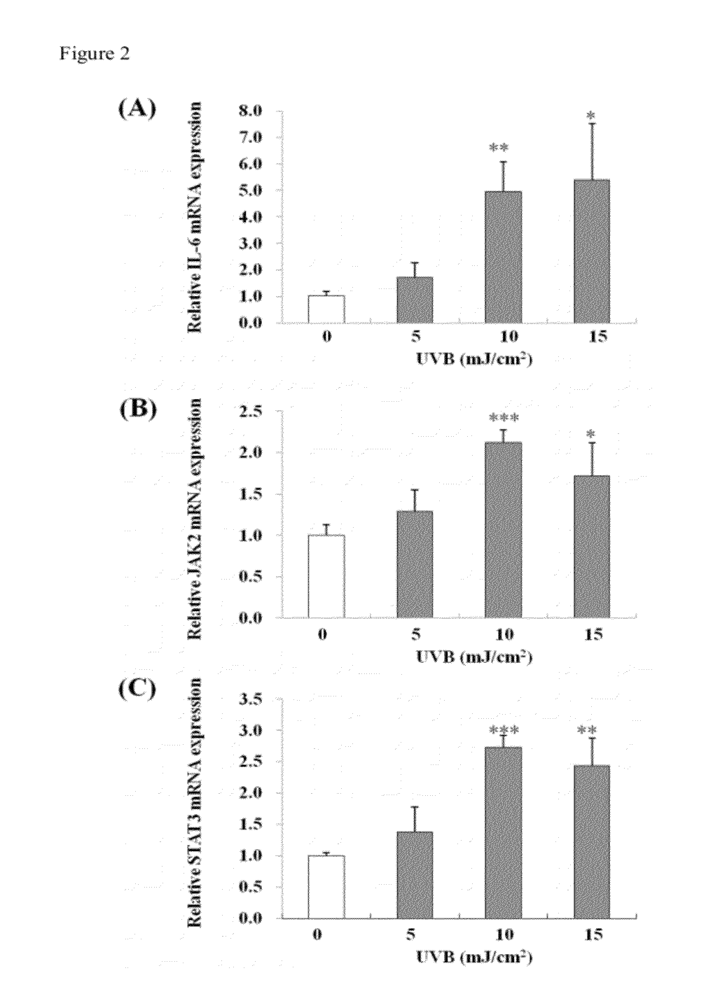

IL-6 binds to cell surface receptors to induce the signal transduction in the JAK2-STAT3 pathway (Sherman C T, Brasier A R. Role of signal transducers and activators of transcription 1 and −3 in inducible regulation of the human angiotensinogen gene by interleukin-6. Mol Endocrinol 2001; 15:441-457). The mRNA abundance of IL-6, JAK2 and STAT3 in response to UVB was detected by the real-time RT-PCR. The UVB radiation dose-dependently (0-15 mJ/cm²) increased IL-6 mRNA at 24 h (FIG. 2A). Consistent to the cell viability experiment, it was found that the maximal mRNA expressions of JAK2 and STAT3 genes were induced at 10 mJ/cm² UVB radiation and the expressions were slightly reduced at 15 mJ/cm² UVB radiation at 24 h after radiation (FIGS. 2 B and 2C).

### Effect of TA on UVB-Induced IL-6 Protein Production

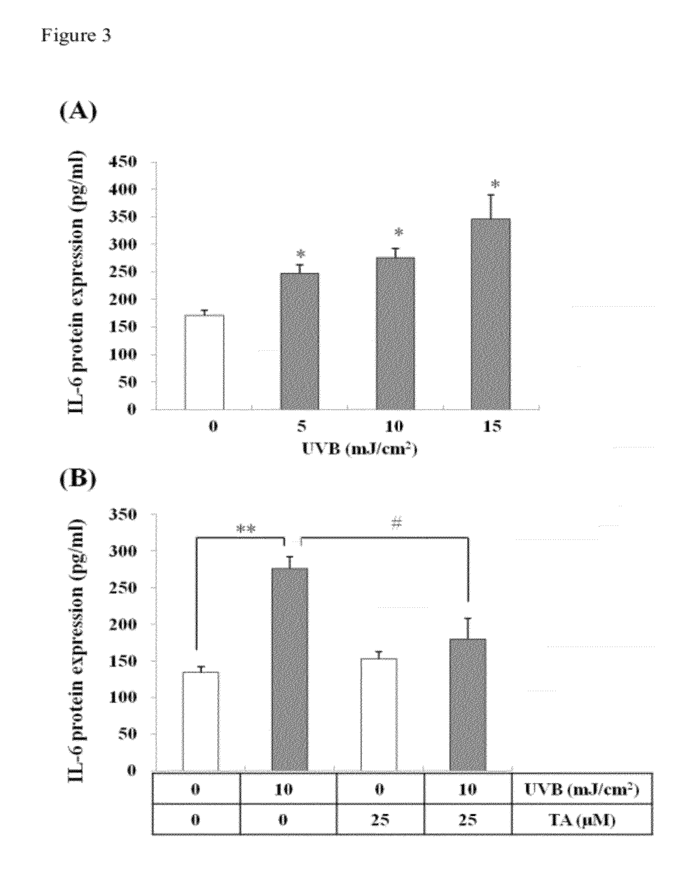

The present invention determined whether UVB can induce IL-6 protein production in the ARPE-19 cell culture supernatants by ELISA. It was found that UVB radiation increased IL-6 protein expression (FIG. 3A). The test of the effect of TA on IL-6 showed that TA attenuated UVB-induced IL-6 protein production by approximately 40% in the ARPE-19 cells (FIG. 3B).

### Effect of TA on UVB-Induced Phosphorylation of STAT3

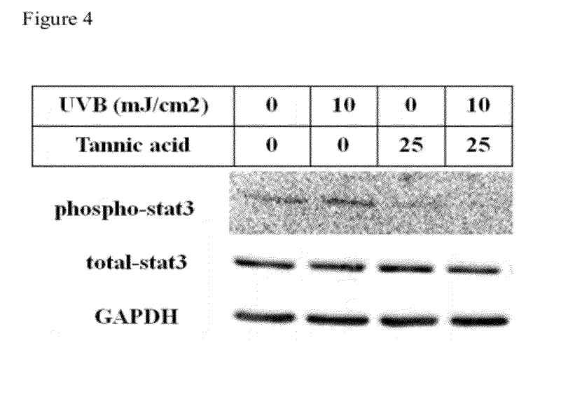

IL-6 binds to its receptor gp130 leading to phosphorylation of Tyr705 of STAT3 (Sherman C T, Brasier A R. Role of signal transducers and activators of transcription 1 and −3 in inducible regulation of the human angiotensinogen gene by interleukin-6. Mol Endocrinol 2001; 15:441-457). The phospho-STAT3^{Tyr705} protein will dimerize and form an activated nuclear transcription complex that will subsequently translocate into the nucleus to regulate the target genes (Sherman C T, Brasier A R. Role of signal transducers and activators of transcription 1 and −3 in inducible regulation of the human angiotensinogen gene by interleukin-6. Mol Endocrinol 2001; 15:441-457). Since UVB can induce IL-6 expression, whether UVB can induce phosphorylation of STAT3 was further tested. Using immunoblotting, it was found that UVB radiation slightly increased STAT3 phosphorylation on Tyr705. The result also showed that TA substantially attenuated UVB-induced STAT3 phosphorylation in RPE (FIG. 4).

Effect of TA or JAK2 Inhibitor on UVB-Induced CFB mRNA

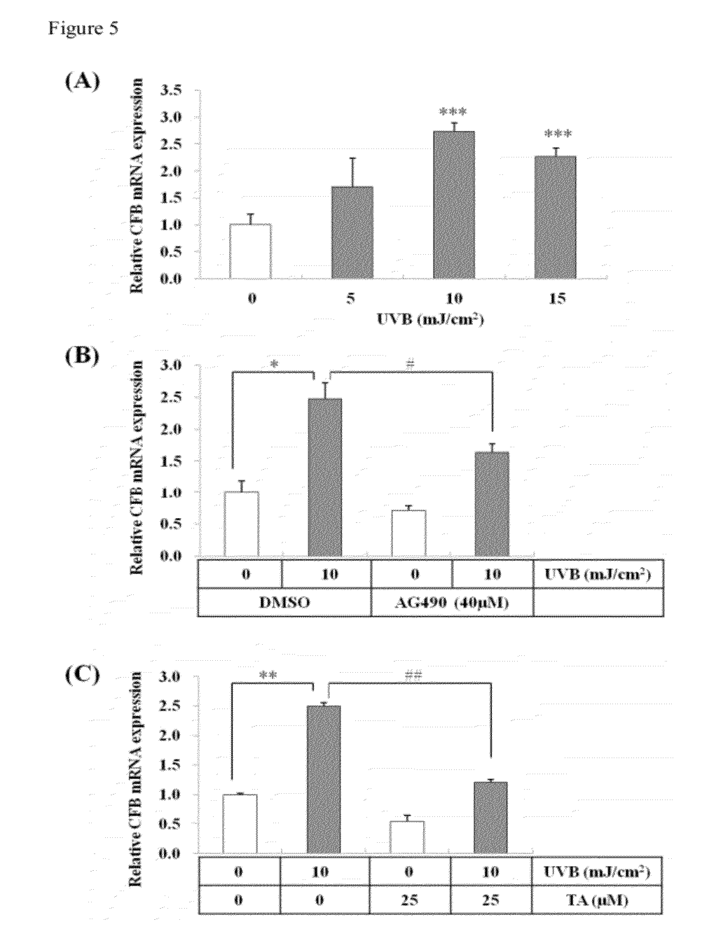

Whether UVB can induce CFB mRNA expression was determined, and the results showed that the maximal CFB mRNA expression was detected at 10 mJ/cm² UVB radiation (FIG. 5A). However, CFB mRNA expression was slightly reduced at 15 mJ/cm² UVB radiation at 24 h after radiation (FIG. 5A). To determine whether UVB-induced CFB up-regulation was via the IL6/JAK2/STAT3 pathway, a specific JAK2 inhibitor, AG490, was used to see the CFB gene expression. AG-490 has been shown to inhibit JAK/STAT signaling and to prevent retinal degeneration after bright light exposure (Samardzija M, Wenzel A, Aufenberg S, Thiersch M, Reme C, Grimm C. Differential role of Jak-STAT signaling in retinal degenerations. FASEB J 2006; 20:2411-2413). Using immunoblotting, it was found that the dose of AG490 (40 μM) did can inhibit the STAT3 phosphorylation on Tyr705 (data not shown), and AG490 also partly attenuated UVB-induced CFB mRNA expression (FIG. 5B). It was also noticed that TA could attenuate UVB-induced CFB mRNA expression in the ARPE-19 cells (FIG. 5C).

One skilled in the art readily appreciates that the present invention is well adapted to carry out the objects and obtain the ends and advantages mentioned, as well as those inherent therein. The cells, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of modulating complement factor B (CFB) expression in cells, comprising administering an effective amount of tannic acid to the cells.

2. The method of claim 1, which is applied in treating or prophylaxis of the disease, disorder or medical condition associated to complement factor B (CFB) expression.

3. The method of claim 2, wherein the disease, disorder or medical condition associated to complement factor B (CFB) expression is selected from age-related macular degeneration, complement factor B deficiency, hemolytic uremic syndrome, atherosclerosis, schizophrenia, glomerulonephritis, autoimmune diseases including Behçet's syndrome, recurrent oral ulceration and Crohn's disease, uveitis, infectious diseases or fungal infection.

4. The method of claim 3, wherein the disease, disorder or medical condition associated to complement factor B (CFB) expression is age-related macular degeneration.

5. The method of claim 1, wherein the complement factor B (CFB) expression is induced by ultraviolet light.

6. The method of claim 5, wherein the ultraviolet light is ultraviolet B.

7. The method of claim 1, wherein the cells are retinal pigment epithelium cells.

8. The method of claim 1, which modulates CFB expression by modulating STAT3 phosphorylation.

9. The method of claim 8, which modulates STAT3 phosphorylation by modulating IL-6 protein production.
